(19) United States
(12) Patent Application Publication
(10) Pub. No.: US 2026/0248387 A1
(43) Pub. Date: Aug. 27, 2026

(54) TABLETOP APPLANATION TONOMETER TO MEASURE THE INTRAOCULAR PRESSURE OF AN EYE

(71) Applicants: Steven E. Maurath, Santa Ana, CA (US); John M. Maggiano, Henderson, NV (US)

(72) Inventors: Steven E. Maurath, Santa Ana, CA (US); John M. Maggiano, Henderson, NV (US)

(21) Appl. No.: 19/061,133

(22) Filed: Feb. 24, 2025

(51) Int. Cl. A61B 3/16

(52) U.S. Cl. CPC A61B 3/16; A61B 2560/0431

(57) ABSTRACT

Disclosed is a tabletop tonometer apparatus that includes an encasement within which a motor controlled applanation tonometer is housed to indicate the intraocular pressure (IOP) of an eye undergoing testing. The tonometer encasement has a head positioning stand on which the head of an individual is positioned to enable the individual's eye to be tested for IOP. A 3-axis (X, Y, Z) tonometer position controller is housed within the tonometer encasement at which to be coupled to the applanation tonometer. The tonometer position controller has three reversible linear motors to generate driving forces for causing tonometer positioning slides interconnected with the applanation tonometer to move in any one or more of three orthogonally aligned directions so that the tonometer moves outwardly from the encasement towards and into contact with the eye of the individual.

### BACKGROUND OF THE INVENTION

### Field of the Invention

This invention relates to a tabletop tonometer apparatus that provides an indication of the intraocular pressure (IOP) of a human eye to be tested at a variety of different locations. The apparatus includes a 3-axis (X, Y, Z) tonometer position controller that is housed within a tonometer encasement at which to move an applanation tonometer in any one or more of three orthogonally aligned directions so as to be properly positioned to enable the eye to be accurately tested for IOP.

### Background Art

Instruments are known that collect data regarding the intraocular pressure (IOP) of a patient's eye to enable medical professionals to access the condition and well-being of the eye. One such instrument that is useful for this purpose is an applanation tonometer which is a non-invasive device that measures pressure or tension in the eye. IOP is measured to provide information to enable the diagnosis and treatment of glaucoma and similar eye diseases. According to a conventional technique, IOP is computed by making a calculation of a contact force as applied by a tip of the applanation tonometer against a patient's cornea and divided by the area of contact. The required force necessary for the tip of the applanation tonometer to applanate the eye of the patient is typically controlled by a physician or a healthcare technician.

However, tonometer testing instruments are typically held in the hand of the health care provider who administers the tests of the patient's eye in a medical office at a time during the patient's visit. What is not known is a desktop or tabletop tonometer apparatus that can be used outside a medical office to allow IOP data to be collected at such locations as a walk-in clinic, a drugstore, or even at a patient's home or office. In this same regard, it would be desirable for the IOP testing to be self-directed by the patient to avoid the need for a medical professional to be present to administer the test and record the test results. Likewise, it would be further desirable for the testing to be completed while the patient is under different physical conditions at different times of the day and at locations that are remote from a medical office. In this case, a variety of test data can be collected and saved to be reviewed later by the medical professional when it is convenient.

### SUMMARY OF THE INVENTION

In general terms, a tonometer apparatus is described comprising a tonometer encasement within which an applanation tonometer is housed and positioned so as to be moved towards and into contact with an individual's eye to be tested for intraocular pressure (IOP). The tonometer encasement is configured to be laid on a flat surface such as the top of a table or desk to enable the individual's IOP to be tested at home or at a medical facility. The encasement includes a head positioning stand with a head rest strap to receive the head of the individual and a chin rest on which the chin of the individual is located. Each of the head rest strap and the chin rest includes an array of pressure sensors that provide signals to a micro-computer to ensure that the head of the individual is properly located on the head positioning stand to enable the eye to be tested. The encasement has an LCD screen at which the IOP test results are displayed. An eye position sensor array is housed within the tonometer encasement to transmit infrared signals towards the individual's eye through infrared transmission slots formed in the encasement. The signals that are reflected from the eye are supplied to the micro-computer to determine if the individual's eye is properly aligned with the applanation tonometer to permit IOP testing. The applanation tonometer is moved towards and away from the eye through a tonometer exit opening that is also formed in the tonometer encasement.

A 3-axis (X, Y, Z) tonometer position controller having a plurality of reversible linear (e.g., stepper) motors is interfaced with the applanation tonometer by which to cause the tonometer to move in any one or more of three orthogonally aligned directions outwardly from the tonometer exit opening so as to contact the individual's eye and achieve full applanation thereof. Each of the motors has an internal encoder that communicates with the micro-computer to enable the position of the applanation tonometer to be determined relative to the eye undergoing testing.

The tonometer position controller includes a pair of vertically upstanding and parallel aligned linear slides that are spaced from one another. A third linear slide extends horizontally between the pair of vertically upstanding linear slides. First and second rotating screws are supported by respective ones of the pair of upstanding linear slides so as to extend vertically upward in the Y-direction of the three orthogonally aligned directions. A pair of vertically positioning tonometer slide plates are coupled to the first and second screws, and the applanation tonometer is interconnected to the pair of slide plates. The first and second rotating screws are driven in tandem by first and second linear motors to cause the pair of vertically positioning tonometer slide plates to move up and down along the screws, whereby the applanation tonometer is correspondingly moved up and down in the Y-direction.

A third rotating screw is supported by the third linear slide so as to extend horizontally in the X-direction of the three orthogonally aligned directions. A horizontally positioning third tonometer slide plate is coupled to the third screw, and the applanation tonometer is interconnected to the third slide plate. The third rotating screw is driven by a third linear motor so as to cause the horizontally positioning third tonometer slide plate to move laterally along the third screw, whereby the applanation tonometer is correspondingly moved side-to-side in the X-direction and horizontally between the pair of upstanding linear slides.

The application tonometer is enclosed by a tonometer housing that lies within the tonometer encasement, and the tonometer housing is attached to a tonometer housing positioning fourth slide plate. A fourth rotating screw extends in the Z-direction of the three orthogonally aligned directions from a fourth linear motor to the fourth slide plate. The fourth rotating screw is driven by the fourth linear motor to cause the tonometer housing positioning fourth slide plate to which the tonometer housing is attached to slide back and forth along a linear tonometer attachment mount. Accordingly, the applanation tonometer is moved with the tonometer housing in the Z-direction outwardly and inwardly relative to the tonometer encasement towards and away from the eye of the individual undergoing testing.

### BRIEF DESCRIPTION OF THE DRAWINGS

FIG. 1 shows a tonometer encasement of a tabletop tonometer apparatus within which an applanation tonometer is housed for testing the eye of an individual for intraocular pressure;

FIG. 2 shows the tonometer encasement of FIG. 1 with the head of the individual located on a head positioning stand of the encasement;

FIG. 3 is illustrative of an eye target position sensor array housed within the tonometer encasement for determining if the head of the individual is positioned on the head positioning stand of FIG. 2 to enable accurate testing of the individual's eye;

FIG. 4 shows a 3-axis (X, Y, Z) tonometer position controller that is housed within the tonometer encasement at which to move the applanation tonometer in any one of three orthogonally aligned directions so as to be positioned to engage the individual's eye to be tested;

FIG. 5 shows a plurality of linear motors of the tonometer position controller of FIG. 4 which are selectively operated to cause respective X, Y and Z-directional linear slides to move the applanation tonometer in any of the 3 orthogonally aligned directions relative to the position of the individual's eye;

FIG. 6 shows a diagrammatic representation of a preferred applanation tonometer to be used to test the individual's eye for intraocular pressure;

FIG. 7 is a block diagram to illustrate the flow of data to a micro-computer that controls the position of the applanation tonometer to ensure that it is properly positioned so as to be in alignment with the individual's eye to be tested for intraocular pressure; and

FIGS. 8A and 8B illustrate the steps by which the tonometer apparatus of this invention tests the eye of the individual for intraocular pressure.

### DESCRIPTION OF THE PREFERRED EMBODIMENT

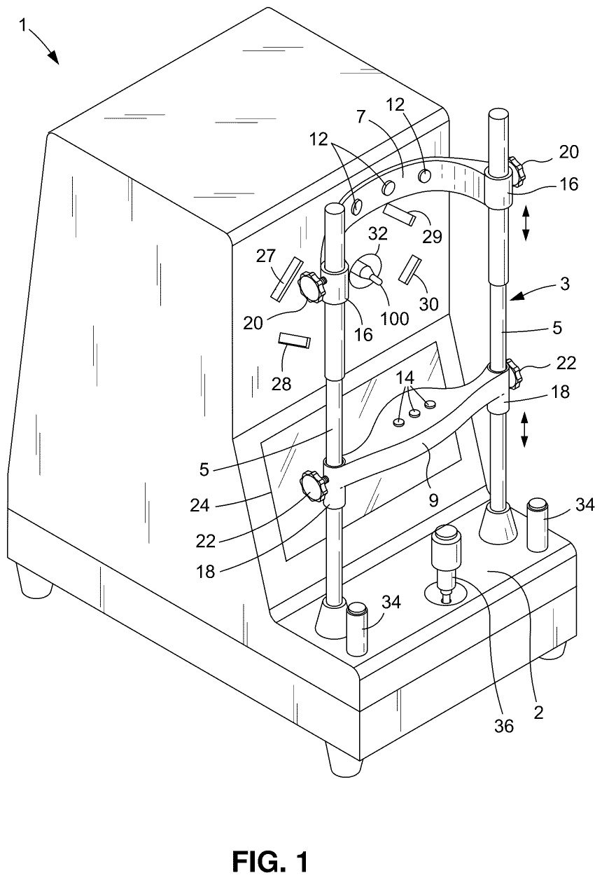

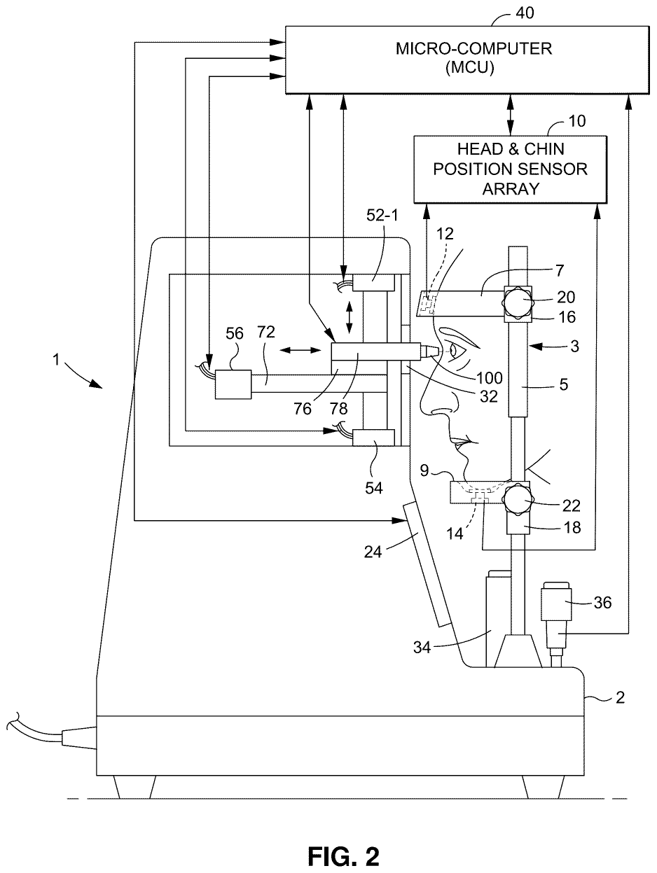

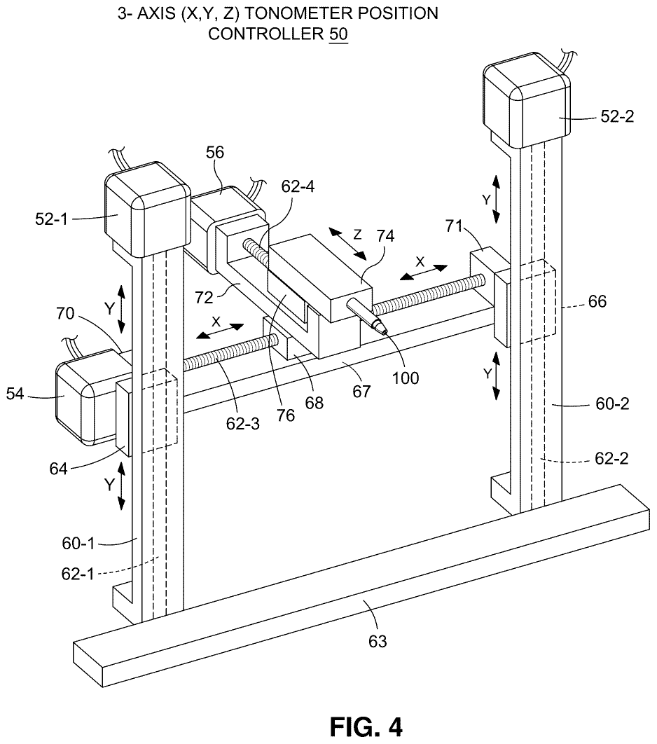

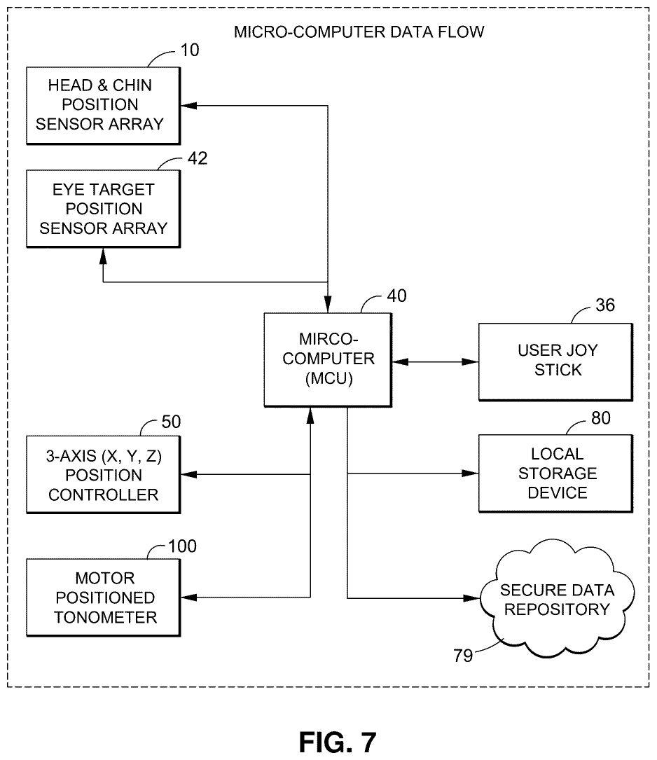

Referring initially to FIGS. 1 and 2 of the drawings, there is shown a tonometer encasement 1 within which a motor controlled applanation tonometer (designated 100 and best shown in FIG. 6) is housed. As will be described in greater detail hereinafter, the applanation tonometer 100 is adapted to measure the intraocular pressure (IOP) of the human eye at a medical facility or at the home or office of an individual. As will also be described, the encasement 1 surrounds a 3-axis (X, Y, Z) tonometer position controller (designated 50 and best shown in FIG. 4) by which the applanation tonometer 100 is displaced in any one or more of three orthogonally aligned directions so as to be accurately aligned with and moved into contact against the individual's eye to be tested for IOP.

Located at the front of the tonometer encasement 1 is a horizontal base 2 to which a head positioning stand 3 is connected. The head positioning stand 3 includes a pair of vertically upstanding head placement guide rails 5 that are spaced from one another. A curved head rest strap 7 at which to receive the individual's head thereagainst extends horizontally between the pair of guide rails 5 at the top of the encasement 1. A chin rest 9 on which to receive the individual's chin extends horizontally between the pair of guide rails 5 below the head rest strap 7.

The head rest strap 7 and the chin rest 9 carry a head and chin position sensor array (designated 10 in FIG. 2) by which to indicate the location of the individual's head relative to the tonometer encasement 1. The sensor array 10 includes a plurality of (e.g., three) forehead pressure sensor pads 12 that are spaced from one another along the head rest strap 7 and a plurality of (e.g., three) chin pressure sensor pads 14 that are spaced from one another along the chin rest 9.

The opposite ends of the headrest strap 7 and the chin rest 9 are coupled to the pair of upstanding head placement guide rails 5 of the head positioning stand 3 by respective pairs of position adjusting sleeves 16 and 18. The position adjusting sleeves 16 and 18 are slidable up and down along the guide rails 5 to correspondingly adjust the position of the headrest strap 7 and the chin rest 9 until the head of the individual is comfortably located and positioned on the head positioning stand 3 above the horizontal base 2 of the tonometer encasement 1 to enable IOP testing of the individual's eye. First and second pairs of locking knobs 20 and 22 are rotated to either tighten or loosen the coupling of the pair of positioning adjusting sleeves 16 and 18 with respect to the pair of guide rails 5. That is, each of the locking knobs 20 and 22 has a locking screw extending therefrom to be moved through respective ones of the position adjusting sleeves 16 and 18 and into and out of engagement with the guide rails 5.

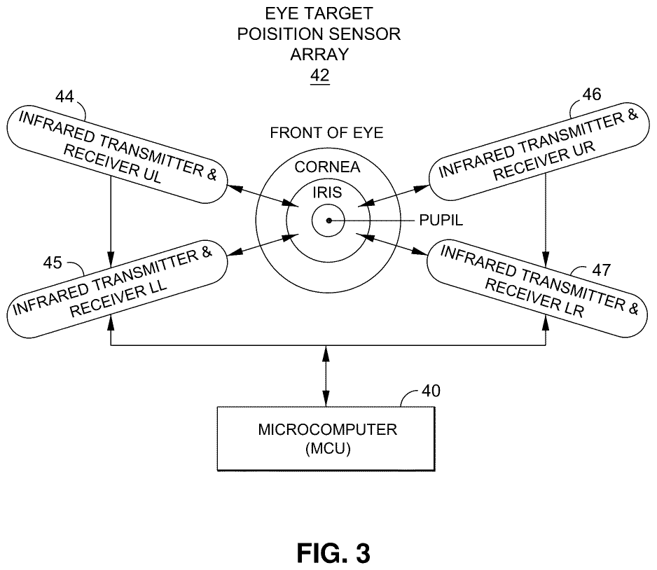

Visible at the front of the tonometer encasement 1 above the horizontal base 2 is an LCD screen 24 at which the IOP test results are displayed to the health care provider and/or the individual undergoing testing. Located at the front of the encasement 1 above the LCD screen are a plurality of (e.g., four) infrared (IR) signal transmission slots 27-30. As will be described when referring to FIG. 3, each of the IR signal transmission slots 27-30 is aligned with a respective infrared transmitter and receiver from an eye target position sensor array 42 that is housed within the tonometer encasement 1. The eye target position sensor array 42 enables the position of the individual's eye to be accurately determined relative to the position of the applanation tonometer 100 that is to be moved towards and into contact with the eye during testing for IOP.

A tonometer exit opening 32 is formed in the front of the tonometer encasement 1 between the IR signal transmission slots 27-30. As will be further described while referring to FIG. 4, the 3-axis (X, Y, Z) tonometer position controller 50 moves the applanation tonometer 100 in any of three orthogonally aligned directions so as to be positioned to move through the tonometer exit opening 32 and towards or away from the individual's eye to be tested.

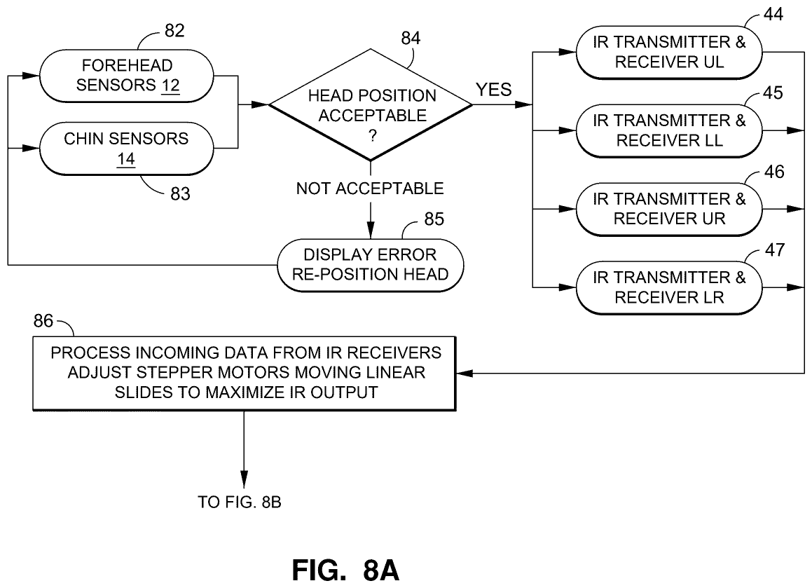

Standing upwardly from the horizontal base 2 of the tonometer encasement 1 alongside the guide rails 5 of the head positioning stand 3 are a pair of hand grips 34. To help the individual's head to be comfortably positioned on the head positioning stand 3, the individual can grasp the hand grips 34 and apply pushing and pulling forces thereto as needed. Also standing upwardly from the horizontal base 2 at the front of the encasement 1 is a joystick 36. A similar joystick (not shown) can be located at the rear of the encasement 1 to be accessible to a healthcare provider. As will be described while referring to FIG. 7, the joystick 36 may be manually manipulated by the individual to control the positioning and movement of the applanation tonometer 100 in the aforementioned three orthogonally aligned directions relative to the eye to be tested. In this case, the joystick 36 is used in substitution of the 3-axis tonometer position controller 50 of FIG. 4.

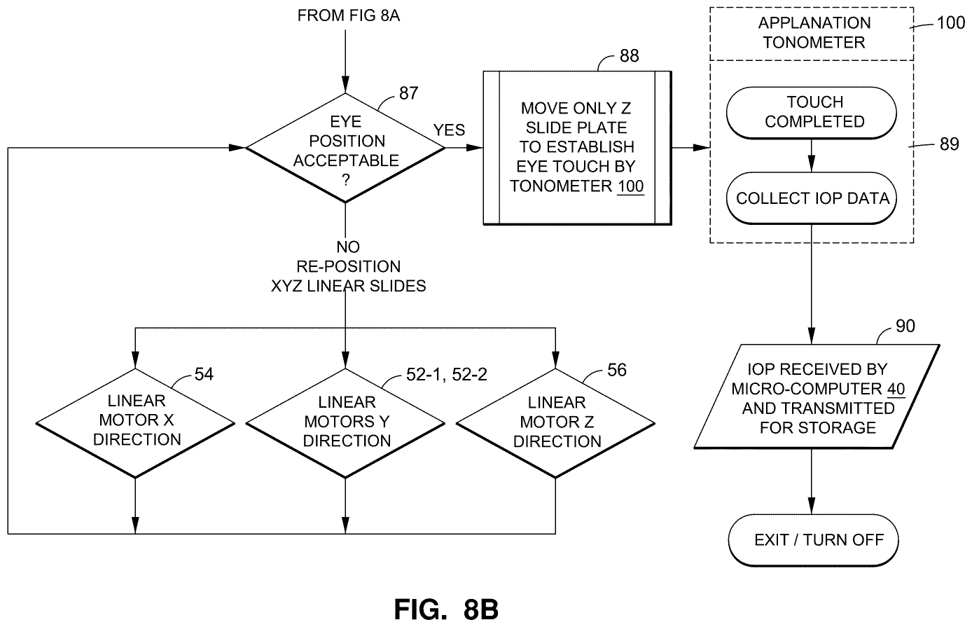

As is best shown in FIGS. 2 and 7, digital representations of the pressures detected by the forehead pressure sensing pads 12 and the chin pressure sensing pads 14 of the head and chin position sensor array 10 are supplied to a micro-computer (MCU) 40 that is housed within the encasement 1. Depending upon the data collected, the micro-computer 40 verifies that the head of the individual is properly positioned against the head rest strap 7 and the chin rest 9 of the head positioning stand 3 to enable the individual's eye to undergo IOP testing. If the outputs of the pressure sensing pads 12 and 14 indicate that the individual's head is not properly positioned for testing, the testing process is aborted and started over again after the head of the individual is properly repositioned on the stand 3 (best represented in FIG. 8A).

FIG. 3 of the drawings shows an eye target position sensor array 42 that is housed within the tonometer encasement 1 to enable the individual's eye to be centered with respect to the position of the applanation tonometer 100 in order to be accurately tested for IOP. The eye target positioning sensor array 42 uses transmitted and reflected infrared (IR) data signals to determine if the individual's eye is stationary prior to being tested and in proper axial alignment with the tonometer 100.

In particular, the position sensor array 42 includes an upper left (UL) and a lower left (LL) infrared transmitter and receiver pair 44 and 45 that transmits input IR signals to and receives reflected IR data signals back from the left side of the iris at the front of the individual's eye. The eye target position sensor array 42 also includes an upper right (UR) and a lower right (LR) infrared transmitter and receiver pair 46 and 47 that transmits input IR signals to and receives reflected IR data signals back from the right side of the iris at the front of the individual's eye. Each infrared transmitter and receiver 44-47 is aligned with a respective one of the infrared signal transmission slots 27-30 that are formed through the front of the tonometer encasement 1.

As is best shown in FIGS. 3 and 7, eye position data that is collected by the left and right side transmitter and receiver pairs 44, 45 and 46, 47 of sensor array 42 is supplied to the micro-computer 40. Should it be determined by the micro-computer 40 that the center of the individual's eye is moving or is not axially aligned with the applanation tonometer 100, the test is temporarily halted. In this case, the optionally used joystick 36 of FIGS. 1 and 2 can be manipulated to slightly adjust the position of the tonometer relative to the individual's eye.

FIG. 4 of the drawings shows the 3-axis (X, Y, Z) tonometer position controller 50 having four reversible linear motors 52-1, 52-2, 54, and 56 which cause the applanation tonometer 100 to move in any of the aforementioned three orthogonally aligned X, Y, and Z-directions with respect to the individual's eye undergoing testing. By way of example only, the linear motors are preferably stepper motors. As will now be explained, the linear motors 52-1, 52-2, 54, and 56 of the position controller 50 are selectively operated to cause the tip of the applanation tonometer 100 to move outwardly from the tonometer encasement 1 through the tonometer exit opening 32 that is formed in the front of the tonometer encasement 1 towards and into momentary contact with the individual's eye until full application is achieved. Once contact is made and full application is achieved, the direction of the reversible linear motors 52-1, 52-2, 54 and 56 is reversed such that the tonometer 100 moves away from the individual's eye to be retracted inwardly through the exit opening 32 to a default safe position within the encasement 1.

First and second linear motors 52-1 and 52-2 are located on upturned top ends of respective ones of a pair of U-shaped Y-directional linear slides 60-1 and 60-2. The linear slides 60-1 and 60-2 stand vertically upward from a horizontal base 63 of the position controller 50 so as to be held in spaced parallel alignment with one another. A first rotating screw 62-1 runs from the motor 52-1 at the upturned top end of the linear slide 60-1 through a vertically positioning first tonometer slide plate 64 that lies adjacent the linear slide 60-1 to an upturned bottom end of the slide. The vertically positioning first tonometer slide plate 64 is adapted to ride along the linear slide 60-1 and move up and down on the first screw 62-1 in the Y-direction indicated in FIG. 4 in response to the first motor 52-1 being operated to cause a rotation of the screw 62-1.

A second rotating screw 62-2 runs from the motor 52-2 at the upturned top end of the Y-directional linear slide 60-2 through a vertically positioning tonometer slide plate 66 that lies adjacent the linear slide 60-2 to an upturned bottom end of the slide. The vertically positioning second tonometer slide plate 66 is adapted to ride along the linear slide 60-2 and move up and down on the second screw 62-2 in the Y-direction in response to the second motor 52-2 operating to cause a rotation of the screw 62-2. An identical U-shaped X-directional linear slide 67 is connected between the first and second tonometer slide plates 64 and 66 as to extend horizontally between the pair of Y-directional linear slides 60-1 and 60-2. The micro-computer 40 of FIG. 7 causes the first and second linear motors 52-1 and 52-2 to operate in tandem so that the vertically positioning first and second tonometer slide plates 64 and 66 move simultaneously up and down in the Y-direction on the first and second screws 62-1 and 62-2.

The third 54 of the four reversible linear motors is attached to an upturned end 70 of the U-shaped X-directional slide 67. A third rotating screw 62-3 runs from motor 54 through the upturned end 70 and a horizontally positioning third tonometer slide plate 68 to the opposite upturned end 71 of linear slide 67. The horizontally positioning third tonometer slide plate 68 is adapted to move side-to-side on the third screw 62-3 and horizontally between the pair of linear slides 60-1 and 60-2 in the X-direction indicated in FIG. 4 in response to the third motor 54 being operated to cause a rotation of the screw 62-3. The upturned opposite ends 70 and 71 of the X-directional linear slide 67 are connected to the vertically positioning first and second tonometer slide plates 64 and 66 so as to move up and down therewith in the Y-direction when slide plates 64 and 66 move along screws 62-1 and 62-2 in response to the operation of motors 52-1 and 52-2.

A U-shaped linear tonometer attachment mount 72 is attached to the horizontally positioning third tonometer slide plate 68 to move side-to-side therewith and laterally between the Y-directional linear slides 60-1 and 60-2 when slide plate 68 moves along screw 62-3 in the X-direction. A tonometer housing 74 is attached to a tonometer housing positioning fourth slide plate 76, and the tonometer housing slide plate 76 is slidable back and forth along the tonometer attachment mount 72 in the Z-direction indicated in FIG. 4. The upturned opposite ends of the U-shaped tonometer attachment mount 72 limit the travel of the tonometer slide plate 76 therealong.

The applanation tonometer 100 is surrounded by an enclosed within the tonometer housing 74 such that the sensing tip of the tonometer projects outwardly from housing 74. The fourth 56 of the four reversible linear motors is connected to a first of the upturns ends of the tonometer attachment mount 72. A fourth rotating screw 62-4 that is connected at one end thereof to the motor 56 extends between the upturned opposite ends of the linear tonometer attachment mount 72. The screw 62-4 runs through the tonometer housing positioning fourth slide plate 76 to which the tonometer housing 74 and the applanation tonometer 100 carried thereby are attached. The tonometer housing positioning fourth slide plate 76 is adapted to slide back and forth along the tonometer attachment mount 72 in the Z-direction in response to the fourth motor 56 being operated to cause a rotation of the fourth screw 62-4.

As the tonometer housing positioning fourth slide plate 76 moves back and forth in the Z-direction, the tonometer housing 74 is moved therewith such that the applanation tonometer 100 surrounded by the housing 74 is correspondingly moved in the Z-direction in or out of the tonometer encasement 1 by way of the exit opening (designated 32 in FIG. 1) thereof. Moreover and being that the horizontally positioning tonometer slide plate 68 is attached to the tonometer attachment mount 72 and the attachment mount 72 is connected to the tonnometer housing positioning slide plate 76 by way of the third screw 62-4, the side-to-side movement of the slide plate 68 along the screw 62-3 causes a corresponding horizontal movement of the tonometer 100 in the X-direction between the Y-directional linear slides 60-1 and 60-2.

As was previously explained, the motors 52-1 and 52-2 are operated to cause the first and second screws 62-1 and 62-2 to rotate and the vertically positioning first and second tonometer slide plates 64 and 66 to move up and down along the screws 62-1 and 62-2 in the Y-direction. Inasmuch as the upturned ends 70 and 71 of the X-directional linear slide 67 between which the third screw 62-3 extends are connected to respective ones of the vertically positioning first and second tonometer slide plates 64 and 66, a movement of the slide plates 64 and 66 up and down in the vertical Y-direction causes a corresponding up and down movement of the tonometer housing 74 and the applanation tonometer 100 surrounded thereby in the Y-direction.

Referring to FIG. 6 of the drawings, there is illustrated a representation of the motor controlled applanation tonometer 100 that is housed within the tonometer encasement 1 shown in FIGS. 1 and 2. As previously explained, the applanation tonometer 100 is moved by the motors of the 3-axis tonometer position controller 50 of FIG. 4 outwardly through the tonometer exit opening 32 formed in the encasement 1 towards and into contact with the eye of the individual until full applanation is achieved and the IOP of the eye is measured. By way of a preferred embodiment, the applanation tonometer 100 of FIG. 6 is the applanation tonometer that is shown and described in our U.S. Pat. No. 9,232,892 issued Jan. 12, 2016, the details of which are incorporated herein by reference.

Briefly, the applanation tonometer 100 includes a prism 102 having a tapered contact tip 104 that lightly touches the eye undergoing testing. When full applanation of the eye is achieved, a first portion of incident light is transmitted in a first direction from a light source (e.g., a laser) 106 through the prism 102 to be absorbed by the cornea. The remaining portion of light is reflected from the contact tip 104 in an opposite direction outwardly through the prism 102 for receipt first by a force responsive piezo disk 108 and then by a beam splitter 110. The reflected light is directed by the beam splitter 110 to a photo detector (e.g., diode) 112 that is responsive to the light being reflected by the contact tip 104 depending upon the area covered by the tip. The outputs of the piezo disk 108 and the photo detector 112 of the applanation tonometer 100 generate force and area data pairs which are supplied to the micro-computer 40 (of FIG. 7) by way of a Bluetooth communication path or the like for IOP processing.

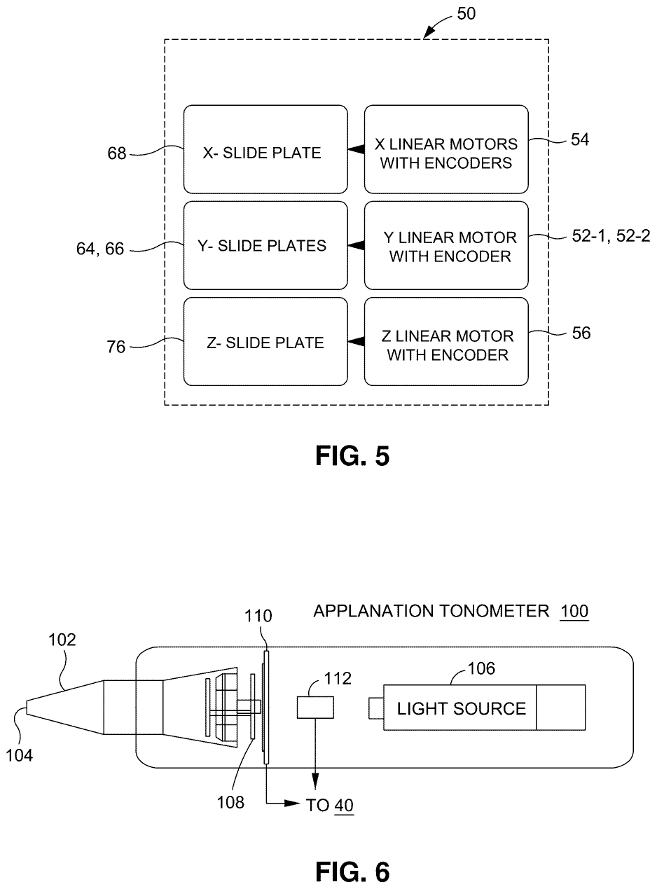

Referring to FIG. 7 of the drawings, the operation and reversible directions of the linear motors 52-1, 52-2, 54, and 56 of the three-axis tonometer position controller 50 of FIG. 4 are shown being controlled by the micro-computer 40 after it first takes into account the head and eye position data of the individual that is provided by the head and chin position sensor array 10 of FIG. 1 and the eye target position sensor array 42 of FIG. 3. Referring briefly to FIG. 5 of the drawings, each of the linear motors 52-1, 52-2, 54, and 56 of the tonometer position controller 50 is shown having a conventional rotary encoder that is responsive to the position of the applanation tonometer 100 as it is moved by the X, Y, and Z-directional slides 52, 54, and 56 relative to the eye of the individual in the aforementioned three orthogonally aligned directions. Position information is supplied by the encoders to the micro-computer 40 so that the linear motors can be selectively operated as required to cause the motor positioned tonometer 100 to be moved in any of the X, Y and Z-directions in order to be accurately aligned with the eye to be tested.

Returning to FIG. 7, the data that is collected by the applanation tonometer 100 is initially transmitted to the micro-computer 40. The data is then analyzed by the micro-computer 40 to ensure data integrity after which the data is routed to a secure data repository 79 where it can be transferred to the cloud to be further reviewed by a health care professional at a time and place that is convenient to the parties. The data, which may result in an IOP display, can then be studied by an attending physician, for example, by way of the internet to permit the condition of the eye of the patient to be diagnosed. Likewise, the data collected by the tonometer 100 can also be transmitted from the micro-computer 40 to the individual being tested at a local storage device 80 such as, for example, a removable memory card or by way of artificial intelligence (AI) human voice technology.

Turning to FIGS. 8A and 8B of the drawings, the steps are described by which the tonometer apparatus disclosed herein is used to test the eye of the individual for intraocular pressure. Referring initially to FIG. 8A, steps 82 and 83 represent the forehead pressure sensing pads 12 and the chin pressure sensing pads 14 of the head and chin position sensor array 10 of FIGS. 1 and 2 collecting data corresponding to the position of the individual's head on the head positioning stand 3. If the position of the individual's head is not acceptable for testing (step 84), the micro-computer 40 of FIG. 2 temporarily interrupts the test and displays an error message on the LCD screen 24 (step 85). If and when the individual's head is properly positioned, the eye position data collected by each IR transmitter and receiver 44-47 of the eye target position sensor array 42 of FIG. 3 is supplied to the micro-computer 40 (step 86) which determines if the motor positioned applanation tonometer 100 of FIGS. 6 and 7 is properly aligned to move into contact with and achieve full applanation of the eye undergoing testing.

Referring now to FIG. 8B, if the micro-computer 40 determines that the position of the individual's eye is not acceptable for IOP testing by the applanation tonometer (step 87), one or more of the linear motors 54, 52-1 and 52-2, and 56 of the 3-axis (X, Y, Z) position controller 50 of FIG. 4 are selectively operated. As described above, the operation of the linear motors causes respective ones of the X-directional tonometer slide plate 68, the Y-directional tonometer slide plates 64 and 66, and the Z-directional tonometer slide plate 76 of the tonometer position controller 50 to move the applanation tonometer 100 in corresponding orthogonally aligned X, Y and Z-directions relative to the eye being tested. Provided that the micro-computer 40 determines that the tonometer 100 is properly aligned with the individual's eye, the linear motor 56 is operated to cause the Z-directional tonometer slide plate 76 to move the applanation tonometer 100 back and forth in the Z-direction towards and away from the eye (step 88).

As shown in FIGS. 2 and 7, the applanation tonometer 100 communicates with the micro-computer 40. Once the micro-computer 40 determines that the individual's eye has been touched by the applanation tonometer 100 and fully applanated, the data is collected by the tonometer in the manner described while referring to FIG. 6 (step. 89). The data is then supplied to the micro-computer 40 to be displayed on the LCD screen 24 and transmitted for remote and local data storage 79 and 80 as is shown in FIG. 7 (step 90). The test data can then be examined by the individual at a home or office and/or a health care provider at a medical facility. At this point, the testing process is completed, and the tonometer apparatus may either be turned off or reused later to conduct a new test.

1. A tonometer apparatus comprising:
a tonometer encasement;
an applanation tonometer housed within said tonometer encasement and adapted to measure the intraocular pressure of an eye of an individual to be tested, said applanation tonometer being movable relative to said tonometer encasement towards and away from the eye of the individual; and
a tonometer position controller interfaced with said applanation tonometer within said tonometer encasement and operable to cause said applanation tonometer to move outwardly from said tonometer encasement towards and into contact with the eye of the individual.

2. The tonometer apparatus recited in claim 1, wherein said tonometer encasement is configured to lay flat on a table top surface.

3. The tonometer apparatus recited in claim 1, wherein said tonometer position controller includes at least first and second motors communicating with said applanation tonometer to generate driving forces by which to cause said applanation tonometer to move in respective ones of said first and second orthogonally aligned directions.

4. The tonometer apparatus recited in claim 3, wherein each of said first and second motors is a reversible linear motor.

5. The tonometer apparatus recited in claim 3, wherein said tonometer position controller also includes first and second tonometer positioning slide paths extending in said first and second orthogonally aligned directions and first and second tonometer positioning slides interconnected with said applanation tonometer and moving along respective ones of said first and second tonometer positioning slide paths, said first and second motors being coupled to said first and second tonometer positioning slides to generate said driving forces by which to cause said first and second tonometer positioning slides to move along said first and second tonometer positioning slide paths, whereby said applanation tonometer is correspondingly moved in said first and second orthogonally aligned directions.

6. The tonometer apparatus recited in claim 5, wherein each of said first and second motors has an encoder by which to indicate the positions of said first and second tonometer positioning slides and said applanation tonometer interconnected therewith on said first and second tonometer positioning slide paths.

7. The tonometer apparatus recited in claim 5, wherein one of said first and second tonometer positioning slide paths is a screw along which the first of said first and second tonometer positioning slides is movable, the first of said first and second motors being operable to cause said screw to rotate and the first tonometer positioning slide to ride along said screw, whereby said applanation tonometer is correspondingly moved in the first of said first and second orthogonally aligned directions.

8. The tonometer apparatus recited in claim 7, wherein said screw is held by a vertically upstanding linear screw support such that said first tonometer positioning slide moves vertically up and down on said screw and along said linear screw support in the first of said orthogonally aligned directions.

9. The tonometer apparatus recited in claim 7, further comprising a tonometer housing attached to said first tonometer positioning slide and surrounding said applanation tonometer such that an eye contact tip of said tonometer extends outwardly from said housing, and a linear attachment mount along which said first tonometer positioning slide is movable, said screw extending from said first motor to said first tonometer positioning slide and said first motor being operable to cause said screw to rotate such that said first tonometer positioning slide moves along said screw and the tonometer housing attached to the first tonometer positioning slide moves with said slide along said attachment mount, whereby said applanation tonometer that is surrounded by said tonometer housing is correspondingly moved in the first of said orthogonally aligned directions and the eye contact tip of said tonometer moves back and forth towards and away from the eye of the individual.

10. The tonometer apparatus recited in claim 9, wherein said linear attachment mount has a U-shape with first and opposite upstanding ends to create stops to limit the movement of the first tonometer positioning slide and the tonometer housing attached thereto along said attachment mount in the first of said first and second orthogonally aligned directions, said screw extending between the first and opposite upstanding ends of said U-shaped linear attachment mount.

11. The tonometer apparatus recited in claim 9, wherein said linear attachment mount is attached to the second of said first and second tonometer positioning slides such that said applanation tonometer that is interconnected with said first and second tonometer positioning slides moves in the second of said first and second orthogonally aligned directions when said second tonometer positioning slide rides along the second of said first and second tonometer positioning slide paths.

12. The tonometer apparatus recited in claim 1, wherein said tonometer encasement has a tonometer exit opening formed therein through which said applanation tonometer passes when said tonometer position controller operates to cause said applanation tonometer to move outwardly from said tonometer encasement towards and into contact with the eye of the individual.

13. The tonometer apparatus recited in claim 1, wherein said tonometer encasement includes a head positioning stand having a head rest strap against which the individual's head is positioned and a chin rest against which the user's chin is positioned, each of said head rest strap and said chin rest having an array of pressure sensors mounted thereon to produce output signals in response to the pressure applied thereto by the head and chin of the individual, said output signals indicating the position of the individual's head on said head positioning stand and the position of the individual's eye undergoing testing relative to said applanation tonometer.

14. The tonometer apparatus recited in claim 13, further comprising an eye target position sensor housed within said tonometer encasement and including at least one transmitter and at least one receiver to produce an infrared signal that is transmitted from said transmitter and reflected to the receiver by the eye of the individual undergoing testing, said infrared signal providing an indication of the alignment of the individual's eye with said applanation tonometer.

15. The tonometer apparatus recited in claim 14, further comprising a computer communicating with each of the arrays of pressure sensors mounted on the head rest strap and the chin rest of said head positioning stand and with the transmitter and the receiver of said eye target position sensor, said computer being responsive to the output signals produced by said arrays of pressure sensors and the infrared signal transmitted by said transmitter and reflected to said receiver to indicate the positions of the head and eye of the individual with respect to said applanation tonometer.

16. The tonometer apparatus recited in claim 1, wherein said tonometer position controller includes first, second and third motors operable to cause said applanation tonometer to move in first, second and third orthogonally aligned directions;
first, second and third tonometer positioning slide paths extending in said first, second and third orthogonally aligned directions; and
first, second and third tonometer positioning slides interconnected with said applanation tonometer and being movable along respective ones of said first, second and third tonometer slide paths,
said first, second and third motors being coupled to said first, second and third tonometer positioning slides to generate driving forces for causing said first, second and third slides to ride along said first, second and third tonometer slide paths, whereby said applanation tonometer is correspondingly moved in said first, second and third orthogonally aligned directions towards and into contact with the eye of the individual to be tested.

17. The tonometer apparatus recited in claim 16, wherein each of said first, second and third tonometer positioning slide paths is a screw coupled to one of said first, second and third motors, said first, second and third tonometer positioning slides riding along respective ones of said screws in said first, second and third orthogonally aligned directions in response to the operation of said motors.

18. The tonometer apparatus recited in claim 16, wherein each of said first, second and third motors includes an encoder to indicate the positions of said first, second and third tonometer positioning slides and said applanation tonometer interconnected therewith as said slides ride along said first, second and third tonometer slide paths.

19. The tonometer apparatus recited in claim 18, further comprising a computer interfaced with the encoders of said first, second and third motors, said computer controlling said motors so that said motors are selectively operated to generate said driving forces for causing said first, second and third tonometer positioning slides to move along said first, second and third tonometer positioning slide paths in said first, second and third orthogonally aligned directions depending upon the position of said applanation tonometer relative to the eye of the individual.
